(19) United States
(12) Patent Application Publication
(10) Pub. No.: US 2009/0192220 A1
(43) Pub. Date: Jul. 30, 2009

(54) TREATMENT FOR SOLID TUMORS

(75) Inventors: David William White, Norwell, MA (US); Jin Shengfang, Newton, MA (US); Robert Mark Coopersmith, Chestnut Hill, MA (US); Scott Edward Malstrom, Reading, MA (US); Thi T. Nguyen, Saugus, MA (US)

Correspondence Address: HARNESS, DICKEY, & PIERCE, P.L.C, 7700 Bonhomme, Suite 400, ST. LOUIS, MO, 63105, US

(73) Assignee: Ore Pharmaceuticals Inc., Gaithersburg, MD (US)

(21) Appl. No.: 12/272,145

(22) Filed: Nov. 17, 2008

Related U.S. Application Data
(60) Provisional application No. 60/989,062, filed on Nov. 19, 2007.

(51) Int. Cl. A61K 31/255; A61P 35/00

(52) U.S. Cl. 514/517

(57) ABSTRACT

A method for treating a solid tumor in a subject comprises administering to the subject an ACAT inhibitory compound or a prodrug thereof, for example avasimibe, wherein (a) the solid tumor is at least about 2 mm in diameter and (b) the compound or prodrug thereof is administered in an amount that is therapeutically effective, but ineffective to cause unacceptable toxicity to normoxic tissues.

This application claims the benefit of U.S. provisional application Ser. No. 60/989,062 filed on Nov. 19, 2007, the entire disclosure of which is incorporated herein by reference.

### FIELD OF THE INVENTION

The present invention relates to pharmacotherapy for solid tumors, more particularly such tumors that are malignant. The invention also relates generally to methods for treating conditions induced at least in part by hypoxia, including but not limited to growth of solid tumors.

### BACKGROUND

Acyl coenzyme A: cholesterol O-acyl transferase (ACAT), described as a “ubiquitous intracellular enzyme” (Winum et al. (2004) Expert Opin. Ther. Patents 14:1273-1308), catalyzes acylation of intracellular free cholesterol to form cholesterol esters. Inhibitors of this enzyme represent an established approach to treatment of hypercholesterolemia and inhibition of atherosclerosis. A lead candidate for such use has been the ACAT inhibitor avasimibe (N-2,6-dipropan-2-ylphenoxy)sulfonyl-2-(2,4,6-tripropan-2-ylphenyl)acetamide), previously known as CI-1011. This compound was discontinued in Phase III clinical trials for treatment of atherosclerosis.

There are two isoforms of ACAT in mammals, ACAT1 and ACAT2, which tend to be differentially expressed in different tissues.

Llayerias et al. (2003) Cardiovascular Drug Reviews 21:33-50 have reviewed the pharmacology of avasimibe. They indicate that, although avasimibe has been claimed to exhibit a high degree of selectivity for ACAT2, both isoforms are probably inhibited by administration of 500 mg avasimibe per day to a human subject.

Information on avasimibe as indicated in atherosclerosis and hyperlipidemia has been reviewed by Burnett & Huff (2002) Current Opinion in Investigational Drugs 3:1328-1333.

As reported by de Médina et al. (2004) Curr. Med. Chem.—Anti-Cancer Agents 4(6):491-508, at least one known anti-tumor agent, namely tamoxifen, has been reported to have some affinity for ACAT (K_{d}=6.74 μM), although much weaker than its affinity for the estrogen receptor (K_{d}=1nM).

Kim et al. (1996) J. Antibiotics 49:31-36 isolated the compound GERI-BP-002A from a culture of Aspergillus fumigatus. The compound was reported to inhibit ACAT activity in a rat liver microsome assay and to exhibit cytotoxicity against various cell lines including HCT-15 (a human colon cancer cell line) and A549 (a non-small cell lung cancer cell line).

Ahn et al. (2006) extracted several sesquiterpenes from Ixeris dentata root. One of these, crepiside I, was reported to be cytotoxic to human colon carcinoma and lung adenocarcinoma cells, and to show ACAT inhibitory activity.

Asakuma et al. (2004) J. Urol. 171(4 Suppl.):265-266 reported that ACAT1 was highly expressed in renal cell carcinoma lines, and that the compound SMP-500, said to be an ACAT inhibitor, blocked 5 nM paclitaxel-induced phosphorylation processes implicated in chemoresistance of cancer cells. They proposed that SMP-500 augments chemosensitivity by modulating multi-drug resistance signaling through inhibition of ACAT activity and that the compound may have therapeutic potential for treatment of renal cell carcinoma.

Dessi et al. (2003) Proc. Am. Assoc. Cancer Res. 94(Meet.):323-324 reported that the compound Sandoz 58-035 reduced in vitro proliferation of leukemia cells, and proposed that ACAT inhibitors “may warrant consideration as innovative targeted anticancer agents.”

International Patent Publication No. WO 2005/094864 proposed use of an inhibitor of microsomal triglyceride transfer protein (MTP), HMG-CoA reductase, diacylglycerol acyltransferase (DGAT) or ACAT to prepare a pharmaceutical composition for treatment of tumors, more particularly tumors that secrete Hedgehog and Wnt proteins, which are involved in signaling pathways. It was more particularly proposed therein that activity of these proteins requires that they be lipid-modified, and that any inhibitor of lipid modification of Hedgehog or Wnt proteins can be used to prepare the pharmaceutical composition. Lipid modifications were said to include palmitoylation and cholesterol modification, Hedgehog proteins being palmitoylated and cholesterol modified, but Wnt proteins being palmitoylated only.

Further information on possible effects of lipoprotein-lowering drugs, specifically mentioning statins (HMG-CoA reductase inhibitors) and MTP inhibitors, on Hedgehog and Wnt signaling pathways is presented by Eaton (2006) Curr. Opin. Genetics & Development 16:17-22.

Tosi & Tugnoli (2005) Clinica Chimica Acta 359:27-45, in a review of the role of abnormal cholesterol metabolism, particularly an increase in intracellular cholesteryl esters, in malignancy, mentioned a study in which intracellular activity of ACAT was reportedly higher in clear-cell kidney tumors than in normal kidney tissue. They also remarked that esterification of cholesterol by ACAT has a protective role in cells, since the esters are less cytotoxic than free cholesterol.

D\check{r}ímal et al. (2005) Gen. Physiol. Biophys. 24(4):397-409 tested the ACAT inhibitor VULM-1457 for effects on expression of the proliferative hormone adrenomedullin (AM) in normoxic and hypoxic human hepatoblastoma cell lines. They reported that VULM-1457 down-regulated specific AM receptors in both normoxic and hypoxic cells, and reduced hypoxia-induced AM secretion.

European Patent Application No. EP 1 586 644 proposed that ACAT inhibitors can modulate angiogenesis through an increase in abundance of caveolin-1. It was stated that decrease in angiogenesis would be beneficial in angiogenesis-dependent tumor growth and metastatic disease through drugs that increase intracellular free cholesterol and thereby increase caveolin-1 abundance. It was proposed that increase in caveolin-1 can be achieved with ACAT inhibitors such as avasimibe and others.

Goto et al. (2005) Cancer Letters 219:215-222 reported that the DGAT inhibitor xanthohumol inhibited proliferation of a human fibrosarcoma cell line under hypoxic but not under normoxic conditions. Xanthohumol also reportedly suppressed hypoxia-enhanced motility of cells. The data were said to suggest that lipid metabolism may play an important role for hypoxic tumor cells and propose a new therapeutic target for cancer chemotherapy.

New therapies for inhibition of tumor growth and metastasis are always desired. It would be particularly desirable to identify a therapeutic method that would be effective to inhibit further growth of solid tumors that have already become large enough to be readily detected, for example about 2 mm diameter or larger.

Efforts have been made to discover new uses for avasimibe and other ACAT inhibitors, not limited to their ACAT-inhibitory mode of action. The present invention arises in part from such efforts.

### SUMMARY OF THE INVENTION

It has now surprisingly been found that compounds known to be active as ACAT inhibitors suppress induction of genes for proangiogenic factors such as vascular endothelial growth factor (VEGF), factors promoting cell survival and proliferation such as insulin-like growth factor binding protein 3 (IGF-BP3), and/or pH regulating factors such as carbonic anhydrase 9 (CA9), via a hypoxia-responsive element (HRE) in promoter regions of such genes, in cells under hypoxic conditions. Onset of hypoxia, which is known to occur in a solid tumor that has reached a diameter of about 2 mm, normally promotes angiogenesis and proliferation through activation of HRE, but when activation of HRE is suppressed by a compound as provided herein, it is believed that these hypoxia-induced effects are suppressed, resulting in inhibition of further growth of the tumor.

It has further been found that the beneficial suppressive effects of the present compounds in hypoxic conditions are obtainable at inhibitor concentrations that are not unacceptably toxic (e.g., not unacceptably cytotoxic) in the normoxic conditions that typically occur outside the target tumor. For example, VEGF transcript is not suppressed under normoxia at compound concentrations causing strong VEGF transcript suppression under hypoxia. Avoidance of suppression of VEGF in normoxic conditions is an important benefit of the present compounds over agents that are less specific to hypoxia-induced processes. Specifically, preserving VEGF levels in normoxic tissues is important given the role of VEGF in maintaining normal vascular homeostasis (Lee et al. (2007) Cell 130:691-703, incorporated herein by reference without admission as to its status as prior art or otherwise to the present invention).

The compounds of interest herein are known to have ACAT inhibitory activity, but it is not known whether inhibition of ACAT plays any direct or indirect role in their hypoxia-selective suppressive effects on tumor growth-promoting factors such as VEGF, IGF-BP3 and CA9. Indeed some evidence exists that such effects are not ACAT-mediated. Yet the effects have been seen not only with avasimibe, but also with at least one other compound, TMP-153, that is chemically dissimilar to avasimibe but shares with it an affinity for ACAT.

These results point to a new role for ACAT inhibitory compounds such as avasimibe in treatment of solid tumors of at least about 2 mm diameter, for example by inhibition of tumor growth resulting from inhibition of hypoxia-induced angiogenesis. It will be understood that use herein of the term “ACAT inhibitory compound” to denote a compound useful according to the present invention does not imply that the beneficial effects observed result from inhibition of ACAT. Instead the term is used to define a diverse group of compounds useful herein by means of a functional test (ACAT inhibition) that does not necessarily reflect how the compounds act in vivo.

Accordingly, there is now provided a method for treating a solid tumor in a subject, comprising administering to the subject an ACAT inhibitory compound or a prodrug thereof, wherein (a) the solid tumor is at least about 2 mm in diameter and (b) the compound or prodrug thereof is administered in an amount that is therapeutically effective, but ineffective to cause unacceptable toxicity to normoxic tissues.

There is further provided a method for reducing tumor growth and/or metastasis in a subject having a cancerous or precancerous condition, comprising determining presence of one or more solid tumors having a diameter of at least about 2 mm in tissue of the subject, and if such presence is determined administering to the subject an ACAT inhibitory compound or a prodrug thereof in an amount that is therapeutically effective, but ineffective to cause unacceptable toxicity to normoxic tissues.

There is still further provided a method for treating a hypoxia-induced condition in a subject, comprising administering to the subject an ACAT inhibitory compound or a prodrug thereof, wherein the compound or prodrug thereof is administered in an amount that is therapeutically effective, but ineffective to cause unacceptable toxicity to normoxic tissues.

The above methods are illustrated herein with specific reference to avasimibe, but it will be recognized that the invention is not limited to use of that particular ACAT inhibitory compound.

Other embodiments, including particular aspects of the embodiments summarized above, will be evident from the detailed description that follows.

### BRIEF DESCRIPTION OF THE DRAWINGS

FIG. 1 presents results of a first study, as described in Example 1, showing concentration-dependent inhibition of hypoxia-induced activation of a hypoxia response element (HRE) by avasimibe, as measured by luciferase activity in cells transfected with an HRE-luciferase reporter plasmid.

FIG. 2 presents results of a second study, as described in Example 1, showing concentration-dependent inhibition of hypoxia-induced HRE activation by avasimibe, as measured by luciferase activity in cells transfected with an HRE-luciferase reporter plasmid.

FIG. 3 presents results of a study, as described in Example 2, showing concentration-dependent inhibition of hypoxia-induced HRE activation by avasimibe and TMP-153, as measured by luciferase activity in cells transfected with an HRE-luciferase reporter plasmid.

FIG. 4 presents results of a study, as described in Example 3, showing concentration-dependent inhibition of hypoxia-induced HRE activation by avasimibe and TMP-153 (graph A, HRE-luciferase reporter system) but no such inhibition of non-HRE-mediated events (graph B, cytomegalovirus (CMV)-luciferase reporter system), indicating that effects of these compounds are not related to non-specific effects on gene transcription or post-transcriptional events. Comparative data are included for the heat shock protein 90 (HSP-90) inhibitor 17-AAG (17-(allylamino)-17-demethoxygeldanomycin), which lowered luciferase activity in both systems.

FIG. 5 presents results of a study, as described in Example 4, showing no effect of avasimibe, but a strong negative effect of 17-AAG, on cell health and viability under hypoxic conditions, as measured by cellular ATP charge (labeled “% RLU (ATPLite readout)” in FIGS. 5 and 6).

FIG. 6 presents results of a study, as described in Example 4, showing no effect of avasimibe, but a strong negative effect of 17-AAG, on cell health and viability under normoxic conditions, as measured by cellular ATP charge.

FIG. 7 presents results of a study, as described in Example 5, showing suppression by avasimibe of hypoxia-induced heme oxidase 1 (HO-1) promoter activation in a luciferase reporter transgenic mouse. Hypoxia was induced by cadmium chloride (CdCl₂). Activation is measured by fold change in luciferase activity. Statistical analysis represents comparisons between control and avasimibe treated groups (*p<0.05, **p<0.01).

FIG. 8 presents results of a study, as described in Example 6, showing concentration-dependent suppression by avasimibe of hypoxia-induced induction of VEGF mRNA in HeLa cells. Statistical analysis represents comparisons between samples from hypoxia-induced cells and hypoxia-induced avasimibe treated cells (*p<0.05).

FIG. 9 presents results of a study, as described in Example 7, showing concentration-dependent suppression by avasimibe of hypoxia-induced induction of VEGF protein accumulation in conditioned medium from HeLa cells. In this study hypoxia was induced by deferoxamine (DFX). Statistical analysis represents comparisons between samples from hypoxia-induced control cells and hypoxia-induced avasimibe treated cells (*p<0.05).

FIG. 10 presents results of a study, as described in Example 8, showing suppression by avasimibe of VEGF mRNA induction under hypoxia but not under normoxia. Comparative data are included for the anti-tumor agent echinomycin, which suppressed VEGF mRNA levels under both hypoxic and normoxic conditions. Statistical analysis represents (1) comparisons between samples from cells grown under normoxic conditions and samples from cells grown under normoxic conditions treated with either avasimibe or echinomycin (*p<0.05), or (2) comparisons between samples from control cells grown under hypoxic conditions and samples from cells grown under hypoxic conditions and exposed to either avasimibe or echinomycin (*p<0.05).

FIG. 11 presents results of a study, as described in Example 10, showing no effect of avasimibe and TMP-153 on hypoxia-induced whole cell accumulation of HIF-1α antigen in A549 cells. Comparative data are included for the phosphoinositide 3 (PI3) kinase inhibitor LY-294002 and the HSP-90 inhibitor 17-AAG, both of which reduced whole cell accumulation of HIF-1α antigen.

FIG. 12 presents results of a study, as described in Example 10, showing no effect of avasimibe and TMP-153 on hypoxia-induced accumulation of HIF-1α antigen in nuclei of A549 cells. Comparative data are included for the PI3 kinase inhibitor LY-294002 and the HSP-90 inhibitor 17-AAG, both of which reduced nuclear accumulation of HIF-1α antigen.

FIG. 13 presents results of a study, as described in Example 11, showing concentration-dependent suppression by avasimibe of VEGF protein accumulation in conditioned medium from colon 26 tumor cells exposed to hypoxic conditions.

FIG. 14 presents results of an in vivo study, as described in Example 12, showing inhibition by avasimibe of solid tumor volume growth in mice. Statistical analysis represents comparisons between vehicle treated animals and avasimibe treated animals (**p<0.005, ***p<0.0005).

FIG. 15 presents results of an in vivo study, as described in Example 12, showing reduction by avasimibe of final tumor weight in mice. Statistical analysis represents comparisons between vehicle treated animals and avasimibe treated animals (*p<0.05, ***p<0.0005).

### DETAILED DESCRIPTION

As described at www.medscape.com/viewarticle/525021_{—}2, for angiogenesis to occur “there must be a switch from the physiologic quintessence of endothelial cells to an ‘angiogenic’ phenotype.” Among metabolic stresses promoting this switch is hypoxia. Tumor neovascularization often lags behind tumor growth, leaving areas of hypoxia. The decrease in oxygen tension in such areas stimulates further angiogenesis through various signaling pathways, via production of numerous transcriptional factors. The most important of these are hypoxia-inducible factors (HIFs), in particular HIF-1 and HIF-2.

The HIF active moiety is a heterodimer of α and β subunits. When oxygen levels are sufficient to meet cellular demand, a condition known as normoxia, HIF-α subunits are subjected to oxygen-dependent prolyl hydroxylation, which promotes an interaction with von Hippel-Lindau (VHL) protein, an E3 ubiquitin ligase. The resulting ubiquitinated HIF-α is rapidly destroyed by proteosomes. On the other hand, when oxygen levels are insufficient, a condition known as hypoxia, prolyl hydroxylation and subsequent HIF-α destruction are avoided, allowing HIF-α to bind to HIF-β at the nuclear HREs of numerous genes, thereby activating expression of these genes. In addition to the prolyl hydroxylation/ubiquitination pathway of degradation, hydroxylation of an asparagme residue in the C-terminal transactivational domain of HIF-α also prevents transcriptional activation. This hydroxylation is catalyzed by FIH (factor inhibiting HIF), which also requires oxygen, thus is restricted or avoided in a condition of hypoxia.

Many genes induced by HIF, and therefore expressed in hypoxic conditions, promote tumor growth in a variety of ways. Of particular interest herein are genes for proangiogenic factors, principally VEGF and its receptors, including FLT1 (VEGF receptor 1) KDR (VEGF receptor 2), neuropilin-1 and neuropilin-2.

Tumor angiogenesis can be assessed in various ways. In one method an antibody to endothelial cells is used to identify tumor vasculature. Microvessel density (MVD) can then be measured as an average over a number of randomly selected areas or in the densest areas of neovascularization (“hotspots”). A variety of immunohistochemical techniques have been used to assess MVD, based on antibodies to endothelial markers such as CD31, CD34 or factor VIII-related antigen.

It has not previously been known that an ACAT inhibitory compound such as avasimibe, at concentrations substantially non-toxic to normoxic tissues, can suppress hypoxia-induced induction of genes for factors promoting tumor growth such as proangiogenic factors. This discovery paves the way for development of novel anti-cancer therapies having an improved side-effect profile by comparison with many current therapies. Not only are adverse effects on off-target normoxic tissues minimized, but it is believed (without being bound by theory) that at least in some tumors the therapeutic effect is multi-faceted, and therefore strengthened or magnified, because inhibition of HRE activation by the ACAT inhibitory compound results in suppression of a plurality of tumor growth-promoting genes that would otherwise be induced by the hypoxic conditions developing in a 2 mm or larger tumor. Among such tumor growth-promoting genes, genes for proangiogenic factors such as VEGF may be especially strongly suppressed.

In various embodiments, therefore, the invention provides a method for treating a solid tumor of at least about 2 mm diameter in a subject. The method comprises administering to the subject an ACAT inhibitory compound or a prodrug thereof in an amount that is therapeutically effective, but ineffective to cause unacceptable toxicity to normoxic tissues.

The subject herein is typically mammalian and can be human or of any nonhuman species, including animal models for human disease and animals requiring veterinary care. A human subject can be, for example, a patient in the care of a physician who may prescribe an ACAT inhibitory compound, e.g., avasimibe, or a prodrug thereof for administration according to the present invention. It is generally desirable that presence of a tumor of at least about 2 mm diameter in tissue of the subject be determined before initiation of treatment according to the present method. Tumors that are non-solid or smaller than about 2 mm diameter are typically not hypoxic and can therefore be expected not to respond well to the present method, which as pointed out above is generally of low toxicity or non-toxic to normoxic tissues.

Any diagnostic technique known in the art that is capable of detecting a 2 mm or larger solid tumor can be used to identify a subject for whom treatment according to the present method can be appropriate. Such techniques include without limitation examining, for example by microscopy with suitable staining, a tissue sample from the subject obtained by excision or biopsy. Alternatively or in addition, an image of tissue obtained by endoscopy, X-ray, CT (computed tomography) or MRI (magnetic resonance imaging) can be examined for evidence of a 2 mm or larger solid tumor.

The term “solid tumor” herein applies to an abnormal mass of tissue that usually does not contain cysts or liquid areas and can arise in any part of the body. Solid tumors may be benign (not cancerous), or malignant (cancerous). Most kinds of cancer other than leukemias can form solid tumors. In general, tumors most suited to treatment by the present method are well-defined as opposed to diffuse masses of tissue and typically have a three-dimensional shape that results in cells in the interior of the tumor becoming hypoxic when the tumor reaches a diameter of about 2 mm. For tumors that are substantially spherical in shape, the term “diameter” is self-explanatory. For tumors that are substantially non-spherical, the term “diameter” herein refers to the size of the tumor in its shortest dimension.

In various embodiments the solid tumor to be treated is benign. In other embodiments the solid tumor to be treated is malignant and may be primary or secondary (metastatic). Solid tumors include, for example, adenocarcinomas, carcinomas, hemangiomas, liposarcomas, lymphomas, melanomas and sarcomas.

The subject having the tumor to be treated has, in some embodiments, a cancerous or precancerous condition, which may be diagnosed or not. Such condition can occur in any organ or body part including, without limitation, the anus, bile duct, bone, bone marrow, brain, breast, cervix, colon, duodenum, esophagus, gallbladder, head and neck, ileum, jejunum, kidney, larynx, liver, lung, mouth, ovary, pancreas, pelvis, penis, pituitary, prostate, rectum, skin, stomach, testes, thyroid, urinary bladder, uterus and vagina.

More particularly, a cancerous or precancerous condition can comprise one or more of the following: acinar adenocarcinoma, acinar carcinoma, acral-lentiginous melanoma, actinic keratosis, adenocarcinoma, adenocystic carcinoma, adenosquamous carcinoma, adnexal carcinoma, adrenal rest tumor, adrenocortical carcinoma, aldosterone-secreting carcinoma, alveolar soft part sarcoma, amelanotic melanoma, ameloblastic carcinoma, ampullary carcinoma, anal canal cancer, anaplastic thyroid carcinoma, angiosarcoma, apocrine carcinoma, Askin's tumor, astrocytoma, basal cell carcinoma, basaloid carcinoma, basosquamous cell carcinoma, biliary cancer, bone cancer, bone marrow cancer, botryoid sarcoma, brain cancer, breast cancer, bronchioalveolar carcinoma, bronchogenic adenocarcinoma, bronchogenic carcinoma, carcinoid, carcinoma en cuirasse, carcinoma ex pleomorphic adenoma, cervical cancer, chloroma, cholangiocellular carcinoma, chondrosarcoma, choriocarcinoma, choroid plexus carcinoma, clear cell adenocarcinoma, colon cancer, colorectal cancer, comedocarcinoma, cortisol-producing carcinoma, cylindrical cell carcinoma, dedifferentiated liposarcoma, ductal adenocarcinoma of the prostate, ductal carcinoma, ductal carcinoma in situ, duodenal cancer, eccrine carcinoma, embryonal carcinoma, endometrial carcinoma, endometrial stromal sarcoma, endometrioid adenocarcinoma, endometrioid carcinoma, epithelioid sarcoma, esophageal cancer, Ewing's sarcoma, exophytic carcinoma, fibroblastic sarcoma, fibrocarcinoma, fibrolamellar carcinoma, fibrosarcoma, follicular thyroid carcinoma, gallbladder cancer, gastric adenocarcinoma, giant cell carcinoma, giant cell sarcoma, giant cell tumor of bone, granulosa cell carcinoma, head and neck cancer, hemangioma, hemangiosarcoma, hepatocellular carcinoma, Hürthle cell carcinoma, ileal cancer, infiltrating lobular carcinoma, inflammatory carcinoma of the breast, intraductal carcinoma, intraepidermal carcinoma, jejunal cancer, Kaposi's sarcoma, Krukenberg's tumor, Kulchitsky cell carcinoma, Kupffer cell sarcoma, large cell carcinoma, larynx cancer, lentigo maligna melanoma, liposarcoma, liver cancer, lobular carcinoma, lobular carcinoma in situ, lung cancer, lymphoepithelioma, lymphosarcoma, malignant melanoma, medullary carcinoma, medullary thyroid carcinoma, meningeal carcinoma, Merkel cell carcinoma, micropapillary carcinoma, mixed cell sarcoma, mucinous carcinoma, mucoepidermoid carcinoma, mucosal melanoma, myxoid liposarcoma, myxosarcoma, nasopharyngeal carcinoma, nodular melanoma, non-clear cell renal cancer, non-small cell lung cancer, oat cell carcinoma, ocular melanoma, oral cancer, osteoid carcinoma, osteosarcoma, ovarian cancer, Paget's carcinoma, pancreatic cancer, papillary adenocarcinoma, papillary carcinoma, papillary thyroid carcinoma, pelvic cancer, periampullary carcinoma, phyllodes tumor, pituitary cancer, pleomorphic liposarcoma, preinvasive carcinoma, primary intraosseous carcinoma, prostate cancer, rectal cancer, renal cell carcinoma, rhabdomyosarcoma, round cell liposarcoma, scar cancer, schistosomal bladder cancer, schneiderian carcinoma, sebaceous carcinoma, signet-ring cell carcinoma, skin cancer, small cell lung cancer, small cell osteosarcoma, soft tissue sarcoma, spindle cell carcinoma, spindle cell sarcoma, squamous cell carcinoma, stomach cancer, superficial spreading melanoma, synovial sarcoma, telangiectatic sarcoma, terminal duct carcinoma, testicular cancer, thyroid cancer, transitional cell carcinoma, tubular carcinoma, tumorigenic melanoma, undifferentiated carcinoma, urachal adenocarcinoma, urinary bladder cancer, uterine cancer, uterine corpus carcinoma, uveal melanoma, vaginal cancer, verrucous carcinoma, villous carcinoma, well-differentiated liposarcoma, Wilm's tumor and yolk sac tumor.

An “ACAT inhibitory compound” herein is any agent that has demonstrated in vitro or in vivo binding affinity for ACAT such that the normal activity of the ACAT enzyme is reduced or eliminated. An ACAT inhibitory compound useful herein can have affinity for other targets (enzymes or receptors) besides ACAT, but in general it is desirable to use an ACAT inhibitory compound having relatively weak or no binding affinity for hormone receptors. “Relatively weak” in the present context means that IC₅₀ or K_{D} of the compound for ACAT is lower than for a hormone receptor.

As illustration, de Médina et al. (2004), supra, reported tamoxifen to have an IC₅₀ of 6.74 μM for ACAT, but an E_{D} of about 1 nM for the estrogen receptor. If used to target hypoxic tumors according to the present method, therefore, such a drug would likely exhibit strong off-target activity that could limit its usefulness according to the present invention.

An ACAT inhibitory compound useful herein can inhibit ACAT1, ACAT2 or both. In one embodiment a compound is selected that is capable of inhibiting both isoforms of the ACAT enzyme, i.e., a dual ACAT1 and ACAT2 inhibitor such as, for example, avasimibe.

ACAT inhibitory compounds most useful herein typically exhibit an IC₅₀ for ACAT1 and/or ACAT2 not greater than about 5 μM, in various embodiments not greater than about 2 μM, about 1 μM, about 500 nM, about 200 nM or about 100 nM. In the present context, it will be understood that different assay techniques can generate different measures of IC₅₀, particularly for a membrane-bound enzyme such as ACAT. For example, avasimibe has been reported as having an IC₅₀ as high as 12 μM and as low as 60 nM (see Llaverías et al. (2003), supra). For the purpose of the present disclosure, IC₅₀ requirements for particular embodiments will be understood to refer to the lowest known IC₅₀ value for a compound reported in the literature (illustratively, in the case of avasimibe, not greater than 60 nM).

In some embodiments the ACAT inhibitory compound administered according to methods of the present invention is an ACAT1 or dual ACAT1/ACAT2 inhibitor exhibiting an IC₅₀ for ACAT1 not greater than about 5 μM, for example not greater than about 2 μM, about 1 μM, about 500 nM, about 200 nM or about 100 nM.

It is again stressed that recitation herein of an IC₅₀ value or range of such values carries no implication that the benefits of the present invention are necessarily mediated by ACAT inhibition. The precise mechanism or mechanisms by which compounds useful herein exert their hypoxia-selective anti-tumor effects remain to be elucidated. Without being bound by theory, one possible explanation for the observed class effect of ACAT inhibitory compounds described herein is that a binding target other than ACAT is involved, having a substantially similar active site to that of ACAT, so that compounds having affinity for ACAT tend also to have affinity for such other binding target.

Many compounds, representing quite varied chemical structure, are reported in the literature to be active as ACAT inhibitory compounds. The following non-limiting list (Table 1) has been assembled from several publicly-accessible sources. Inclusion in Table 1 of a compound or class that might be found not to be a true ACAT inhibitor will not invalidate the inclusion of any other compound or class.

TABLE 1
ACAT inhibitory compounds

| Name or Code No. | Chemical identity or class if available for certain compounds |
|---|---|
| acaterin | 3-(1-hydroxyoctyl)-5-methyl-2(5H)-furanone |
| AD-6591 | |
| avasimibe | N-2,6-dipropan-2-ylphenoxy)sulfonyl-2-(2,4,6-tripropan-2-ylphenyl) |
| | acetamide |
| bezafibrate | |
| AS-183 | |
| AS-186 | |
| BW-447A | |
| CI-976 | 2,2-dimethyl-N-(2,4,6-trimethoxyphenyl)dodecanamide |
| CI-999 | N-(biarylmethyl)urea |
| CL-277082 | 2,4-difluoro-phenyl-N[[4-(2,2-dimethylpropyl)phenyl]methyl]-N- |
| | (heptyl)urea |
| CL-283546 | N′-heptyl-N{[4-(3-methylbutyl)phenyl]methyl}-N(2,4,6-trifluoro- |
| | phenyl)urea |
| CL-283796 | N′-(4-chloro-2,6-dimethylphenyl)-N-heptyl-N-{[4-(3-methylbutyl) |
| | phenyl]methyl}urea) |
| colestyramine | |
| CP-105191 | |
| CP-113818 | |
| crepiside I | |
| crilvastatin | |
| cyclandelate | |
| E-5324 | n-butyl-N′-[2-[3-(5-ethyl-4-phenyl-1H-imidazol-1-yl)propoxy]-6- |
| | methylphenyl]urea |
| EAB-309 | (R)—N-2-(1,3-benzodioxol-4-yl)heptyl-N′-2,6-diisopropylphenylurea |
| eflucimibe | |
| eldacimibe | |
| epicochlioquinone A | |
| F-1394 | (1S,2S)-2-[3-(2,2-dimethylpropyl)-3-nonylureido]cyclohexane-1-yl |
| | 3-[(4R)—N-(2,2,5,5-tetramethyl-1,3-dioxane-4-carbonyl)amino] |
| | propionate |
| F-12511 | (S)-2′,3′,5′-trimethyl-4′-hydroxy-dodecylthioacetanilide |
| FCE-25390 | |
| FCE-27677 | [(−)N-[2,6-bis(1-methylethyl)phenyl]-N′-[(4R,5R)-2-(4-dimethylamino- |
| | phenyl)-4,5-dimethyldioxolan-2-yl]methylurea |
| FCE-28645A | |
| FR-129169 | N-alkyl-N-biphenylylmethyl-N′-arylurea |
| FR-145237 | N-alkyl-N-biphenylylmethyl-N′-arylurea |
| FR-186054 | N-alkyl-N-biphenylylmethyl-N′-arylurea |
| FY-087 | N-[2-[N′-pentyl-(6,6-dimethyl-2,4-heptadinyl)amino]ethyl]-(2-methyl-1- |
| | naphthylthio)acetamide) |
| GERI-BP-001M | |
| GERI-BP-002A | bis(2-hydroxy-3-tert-butyl-5-methylphenyl)methane |
| glibenclamide | |
| glisoprenins | |
| GW-447C88 | |
| gypsetin | |
| ixerin M | |
| K-604 | |
| K-9406 | |
| K-10085 | |
| KW-3033 | 2-bromo-N-(2,6-diisopropylphenyl)-6,11-dihydrodibenz[b,e]oxepin-11- |
| | carboxamide) |
| KY-331 | |
| KY-455 | |
| lateritin | |
| lecimibide | |
| LS-3115 | |
| melinamide | |
| naringenin | |
| NTE-122 | trans-1,4-bis[[1-cyclohexyl-3-(4-dimethylamino phenyl)ureido]methyl] |
| | cyclohexane |
| P-06139 | |
| pactimibe | |
| PD-132301-2 | |
| PD-138142-15 | |
| purpactins | |
| pyripyropenes | |
| R-106578 | |
| RP-64477 | |
| RP-70676 | |
| RP-73163 | alkylsulfinyl diphenylimidazole |
| Sandoz 57-118 | |
| Sandoz 58-035 | 3-[decyldimethylsilyl]-N-[2-(4-methylphenyl)-1-phenethyl] propanamide |
| SC-435 | 1-[4-[4[(4R,5R)-3,3-dibutyl-7-(dimethylamino)-2,3,4,5-tetrahydro-4- |
| | hydroxy-1,1-dioxido-1-benzothiepin-5-yl]phenoxy]butyl]-4-aza-1- |
| | azoniabicyclo[2.2.2]octane methanesulfonate |
| SCH-48461 | (3R,4S)-1,4-bis-(4-methoxyphenyl)-3-(3-phenylpropyl)-2-azetidinone |
| SKF-98016 | |
| SKF-99085 | tetraisopropyl-2-(3,5-di-tert-butyl-4-hydroxyphenyl)ethyl-1,1- |
| | diphosphonate |
| SMP-500 | |
| SMP-797 | 4-aryl-1,8-naphthyridin-2(1H)-on-3-yl urea derivative |
| SR-9223i | tetraisopropyl-2-(3,5-di-tert-butyl-4-hydroxyphenyl)ethyl-1,1- |
| | diphosphonate) |
| T-2591 | ureidophenol derivative |
| tamoxifen | |
| TEI-6522 | N-(7-methoxy-4-oxochroman-8-yl)-2,2-dimethyldodecanamide |
| TEI-6620 | N-[5-(dimethylamino)-2,2,4,6-tetramethyl-2,3-dihydrobenzofuran-7-yl]- |
| | 2,2-dimethyldodecanamide |
| TMP-153 | N-[4-(2-chlorophenyl)-6,7-dimethyl-3-quinolyl]-N′-(2,4-difluorophenyl) |
| | urea |
| TS-962 | |
| U-73482 | |
| U-76807 | |
| ulmoidol | |
| VULM-1457 | 1-(2,6-diisopropylphenyl)-3-[4-(4′-nitrophenylthio)phenyl]urea |
| YM-17E | 1,3-bis[[1-cycloheptyl-3-(p-dimethylaminophenyl)ureido]methyl] |
| | benzene dihydrochloride) |
| YM-750 | 1-cycloheptyl-1-[(2-fluorenyl)methyl]-3-(2,4,6-trimethylphenyl)urea |
| | acyclic (diphenylethyl)diphenylacetamides |
| | N-alkyl-N-biphenylylmethyl-N′-arylureas and derivatives thereof |
| | N-alkyl-N-[(fluorophenoxy)benzyl]-N′-arylureas and derivatives thereof |
| | N-alkyl-N-(heteroaryl-substituted benzyl)-N′-arylureas and derivatives |
| | thereof |
| | 2-(alkylthio)-4,5-diphenyl-1H-imidazole derivatives |
| | amides of 1,2-diarylethylamines and derivatives thereof |
| | N-[1-butyl-4-[3-[3-(hydroxy)propoxy]phenyl]-1,2-dihydro-2-oxo-1,8- |
| | naphthyridin-3-yl]-N′-(2,6-diisopropyl-4-aminophenyl)urea |
| | N-chlorosulfonyl isocyanate and derivatives thereof |
| | 27-cis-p-coumaroyloxyursolic acid |
| | 27-trans-p-coumaroyloxyursolic acid |
| | cyclic sulfides derived from hetero-Diels-Alder reaction of thioaldehydes |
| | with 1,3-dienes |
| | diaryl-substituted heterocyclic ureas and derivatives thereof |
| | N-(2,2-dimethyl-2,3-dihydrobenzofuran-7-yl)amide derivatives |
| | 2-(1,3-dioxan-2-yl)-4,5-diphenyl-1H-imidazoles and derivatives thereof |
| | N-(4,5-diphenylthiazol-2-yl)alkanamides and derivatives thereof |
| | N-(4,5-diphenylthiazol-2-yl)-N′-aryl(thio)ureas and -alkyl(thio)ureas and |
| | derivatives thereof |
| | 2,6-disubstituted-3-imidazolylbenzopyrane derivatives |
| | fatty acid anilides |
| | heterocyclic amides and derivatives thereof |
| | hydroxyphenylurea derivatives |
| | 23-hydroxyursolic acid |
| | indoline derivatives with an amide or urea moiety |
| | N-(4-oxochroman-8-yl)amide derivatives |
| | N-phenyl-6,11-dihydrodibenz[b,e]oxepin-11-carboxamides and |
| | derivatives thereof |
| | polyacetylene analogs |
| | 3-quinolylurea derivatives |
| | short-chain ceramide and dihydroceramide |
| | terpendoles |
| | tetrazole amide derivatives of (+/−)-2-dodecyl-α-p-phenyl-N-(2,4,6- |
| | trimethoxyphenyl)-2H-tetrazole-5-acetamide |
| | 4,4-bis(trifluoromethyl)imidazolines and derivatives thereof |
| | triterpenes and derivatives thereof |

In the case of a compound having an ionizable or salt-forming moiety, it will be understood that mention of such a compound herein encompasses free acid or free base forms of the compound as appropriate as well as pharmaceutically acceptable salts thereof.

A prodrug of an ACAT inhibitory compound can also be used. A prodrug is a compound typically having little or no pharmacological activity itself but capable of releasing, for example by hydrolysis or metabolic cleaving of a linkage such as an ester moiety, an active drug, in this case an ACAT inhibitory compound, upon administration to a subject.

In a particular embodiment, the ACAT inhibitory compound is avasimibe (N-2,6-dipropan-2-ylphenoxy)sulfonyl-2-(2,4,6-tripropan-2-ylphenyl)acetamide), having the formula:

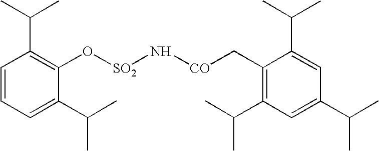

The amount of the ACAT inhibitory compound or prodrug thereof to be administered, and other administration parameters such as frequency and duration of therapy, will be found to depend on the compound or prodrug in question, and on other factors such as the route of administration, formulation of the compound or prodrug, the species, age and body weight of the subject, the nature, location, size, malignancy and invasiveness of the tumor or tumors to be treated, etc. Suitable dosage regimens can be devised for particular circumstances by those of skill in the art, based on the disclosure herein, without undue experimentation. Dosage amounts may be similar to, or different from, those that may have been identified for treatment of dyslipidemia or atherosclerosis.

It is, however, important to balance therapeutic benefit in the form of inhibition of tumor growth with side-effects arising, for example, from inhibition of VEGF activation in normoxic tissues. Doses and other treatment parameters should be selected to avoid an unacceptable degree of such side-effects while providing the desired inhibition of tumor growth. The unexpected discovery that an ACAT inhibitory compound, in hypoxic conditions, can suppress HIF-induced transcript of factors such as VEGF that would otherwise promote tumor growth through angiogenesis and/or other processes, yet not affect production of such factors in normoxic conditions, indicates that the sought-after balance of therapeutic benefit and side-effects is achievable with such a compound. Lack of such balance is a major factor limiting usefulness of more cytotoxic classes of anti-tumor drugs.

Daily doses of an ACAT inhibitory compound likely to be useful herein are typically doses that will provide, at the target tumor site, a compound concentration of about 5 nM to about 50 μM, although in particular cases doses providing lower or higher concentrations may provide effective anti-tumor activity without unacceptable adverse effects in normoxic tissues. Typical daily doses in an animal model can be about 0.05 to about 500 mg/kg body weight; for an adult human patient a suitable daily dose in mg/kg will generally be one that is therapeutically equivalent to such an animal model dose, subject to an appropriate divisor as provided, for example, by the U.S. Food & Drug Administration (FDA) in its “Guidance for Industry: Estimating the Maximum Safe Starting Dose in Initial Clinical Trials for Therapeutics in Adult Healthy Volunteers” (FDA, July 2005), available at www.fda.gov/cder/guidance/index.htm.

An ACAT inhibitory compound or prodrug thereof will normally be administered to an adult human subject at a daily dose in the range of about 0.5 to about 10,000 mg, for example about 1 to about 5000 mg. Illustratively, avasimibe has been shown to be well tolerated by adult human subjects at doses at least up to 750 mg four times daily (i.e., 3000 mg/day). See Kharbanda et al. (2005) Circulation 111:804-807.

It will normally be preferable to administer the compound or prodrug in a dose no greater than necessary to achieve the desired result, to minimize risk of adverse side-effects. A subject, or his/her physician, may elect to start a course of treatment at a low dose, and titrate upwards until a desired degree of anti-tumor efficacy is achieved or until a side-effect is manifested.

“Anti-tumor efficacy” herein refers to an inhibitory effect on tumor growth, which can be manifested as slowing, retarding, arresting or even reversing (i.e., shrinking) growth of the tumor. The present invention is not limited to any particular mode of action; however, it is believed that in some situations the effect on tumor growth will be associated with inhibition of angiogenesis. Failure of the tumor to stimulate development of microvasculature typically results in a marked reduction or cessation of tumor growth, often accompanied by death of cells at least in the hypoxic area of the interior of the tumor. Such inhibition of angiogenesis is believed, without being bound by theory, to be associated with suppression by the ACAT inhibitory compound of hypoxia-induced activation of one or more proangiogenic signaling factors, for example VEGF.

In addition to inhibition of angiogenesis, inhibition of other processes including cell proliferation can occur in some situations. This can be a direct effect of the ACAT inhibitory compound (although not necessarily of ACAT inhibition, as explained above), associated for example with suppression of hypoxia-induced activation of one or more signaling factors, for example IGF-BP3, promoting cell proliferation, or an indirect effect through inhibition of angiogenesis.

Hypoxia-induced activation of signaling factors or cytokines can be involved in pathology of conditions other than growth of solid tumors. In a general embodiment of the present invention, there is accordingly provided a method for treating a hypoxia-induced condition in a subject, comprising administering to the subject an ACAT inhibitory compound or a prodrug thereof, wherein the compound or prodrug thereof is administered in an amount that is therapeutically effective, but ineffective to cause unacceptable toxicity to normoxic tissues.

Methods of the invention can comprise administration of an ACAT inhibitory compound or prodrug thereof by any appropriate route, which can result in local or systemic delivery, or both. Examples of primarily local administration methods suitable in practice of the invention include topical application, local injection and surgical implantation. Examples of primarily systemic administration methods suitable in practice of the invention include oral, rectal, nasal, transmucosal, intrapulmonary, intravenous, intraperitoneal, intramuscular, subcutaneous, intradermal and transdermal administration.

While it can be possible to administer the compound or prodrug unformulated as active pharmaceutical ingredient (API) alone, it will generally be found preferable to administer the API in a pharmaceutical composition that comprises the API and at least one pharmaceutically acceptable excipient. The excipient(s) collectively provide a vehicle or carrier for the API. Pharmaceutical compositions adapted for all possible routes of administration are well known in the art and can be prepared according to principles and procedures set forth in standard texts and handbooks such as those individually cited below.

USIP, ed. (2005) Remington: The Science and Practice of Pharmacy, 21st ed., Lippincott, Williams & Wilkins.

Allen et al. (2004) Ansel's Pharmaceutical Dosage Forms and Drug Delivery Systems, 8th ed., Lippincott, Williams & Wilkins.

Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present invention include, without limitation, solutions, suspensions, powders, granules, tablets, capsules, pills, lozenges, chews, creams, ointments, gels, liposome preparations, nanoparticulate preparations, injectable preparations, enemas, suppositories, inhalable powders, sprayable liquids, aerosols, patches, depots and implants.

Illustratively, in a liquid formulation suitable, for example, for parenteral, intranasal or oral delivery, the API can be present in solution or suspension, or in some other form of dispersion, in a liquid medium that comprises a diluent such as water. Additional excipients that can be present in such a formulation include a tonicifing agent, a buffer (e.g., a tris, phosphate, imidazole or bicarbonate buffer), a dispersing or suspending agent and/or a preservative. Such a formulation can contain micro- or nanoparticulates, micelles and/or liposomes. A parenteral formulation can be prepared in dry reconstitutable form, requiring addition of a liquid carrier such as water or saline prior to administration by injection.

For rectal delivery, the API can be present in dispersed form in a suitable liquid (e.g., as an enema), semi-solid (e.g., as a cream or ointment) or solid (e.g., as a suppository) medium. The medium can be hydrophilic or lipophilic.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. The diluent or diluents selected preferably exhibit suitable flow properties and, where tablets are desired, compressibility.

Lactose, microcrystalline cellulose and starch, either individually or in combination, are particularly useful diluents.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC or hypromellose), hydroxypropyl-cellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

Croscarmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used. Tablets can be uncoated or can comprise a core that is coated, for example with a nonfunctional film or a release-modifying or enteric coating. Capsules can have hard or soft shells comprising, for example, gelatin and/or HPMC, optionally together with one or more plasticizers.

A pharmaceutical composition useful herein typically contains the API in an amount of about 1% to about 99%, more typically about 5% to about 90% or about 10% to about 60%, by weight of the composition. A unit dosage form such as a tablet or capsule can conveniently contain an amount of the compound providing a single dose, although where the dose required is large it may be necessary or desirable to administer a plurality of dosage forms as a single dose. Illustratively, a unit dosage form can comprise the compound in an amount of about 1 to about 800 mg, for example about 5 to about 750 mg or about 10 to about 600 mg.

For oral administration, conventional unit dosage forms such as tablets or capsules, including commercially available dosage forms, are generally suitable for use according to the present methods. Alternatively, dosage forms more specifically adapted to the present use can be developed.

Compounds useful herein can alternatively be delivered to a target site by surgical implantation into an area affected by a tumor, with or without surgical excision of the tumor. Implantable compositions can comprise an ACAT inhibitory compound or prodrug in a biodegradable polymer matrix. A method for delivery of an anticancer drug after surgical resection is described, for example, by Fleming & Saltzman (2002) Clin. Pharmacokinetics 41:403-419, and can be adapted for use herein. Implantation therapy with an ACAT inhibitory compound or prodrug, optionally together with one or more additional drugs, can be combined, if desired, with one or more of surgery, radiotherapy, chemotherapy and immunotherapy. Implants typically provide sustained release of the drug or prodrug over an extended period, for example about 7 days to about 100 days.

A biodegradable polymer useful in preparation of an implantable composition useful herein can comprise any polymer or copolymer that, upon degradation, can dissolve in interstitial fluid without unacceptable adverse effect or toxicity. Certain polymers or monomers from which such polymers are synthesized are approved by the U.S. Food and Drug Administration (FDA) for implantation into humans. A copolymer comprising monomers having different dissolution properties can provide control of dynamics of degradation, for example by increasing the proportion of one monomer over another to control rate of dissolution.

Other delivery systems providing extended release of a drug are also available and adaptable to use with an ACAT inhibitory compound or prodrug. Such systems include, for example, nanoparticulate systems that can provide sustained and targeted delivery of a drug within or in close proximity to a tumor.

Administration of an ACAT inhibitory compound or prodrug thereof according to the present method can take the form of monotherapy (i.e., where no other drug is used concomitantly with the ACAT inhibitory compound or prodrug in treatment of a solid tumor or a cancerous or precancerous condition with which such tumor is associated). In many situations, however, it may be desirable to administer the ACAT inhibitory compound or prodrug in combination therapy, for example as a component of an anti-cancer regimen further comprising one or more of surgery, radiation therapy or administration of one or more drugs other than an ACAT inhibitory compound or prodrug thereof. Selection of other components of the regimen will depend on the type of cancer and its location in the body, the stage of development and aggressiveness or invasiveness of the cancer, and other factors, as will be evident to those of skill in the art.

### EXAMPLES

The following working examples are illustrative of the present invention, and are not to be construed as limiting in any way. The examples illustrate the invention using cellular analysis and in vivo mouse models to demonstrate utility and efficacy of methods of the invention.

### Example 1

### Avasimibe can Suppress Hypoxia-Induced IRE Activation

To demonstrate that avasimibe can suppress or repress the normal cellular response to hypoxia, a luciferase reporter plasmid containing 5 copies of the defined genetic regulatory hypoxia response element (HRE) from the VEGF promoter (Liu et al. (1995) Circ. Res. 77:638-643) was utilized to detect hypoxia-induced HRE activation. For these studies, A549 cells were transfected with HRE-luciferase reporter plasmid, and 24 h later the cells were seeded in 96-well clear-bottom plates at a density of 1.0×10⁵/ml. Avasimibe was added at the indicated concentrations and the cells were then exposed for 20 h to a hypoxic biological atmosphere containing 5% CO₂ and 1% O₂ using a Modular Incubator Chamber (Billups-Rothenberg, Inc.). Cells were then lysed and extracts prepared using the Bright Glow Luciferase Assay System (Promega, Inc.) and luciferase activity measurements were obtained in a Veritas luminometer (Promega, Inc.).

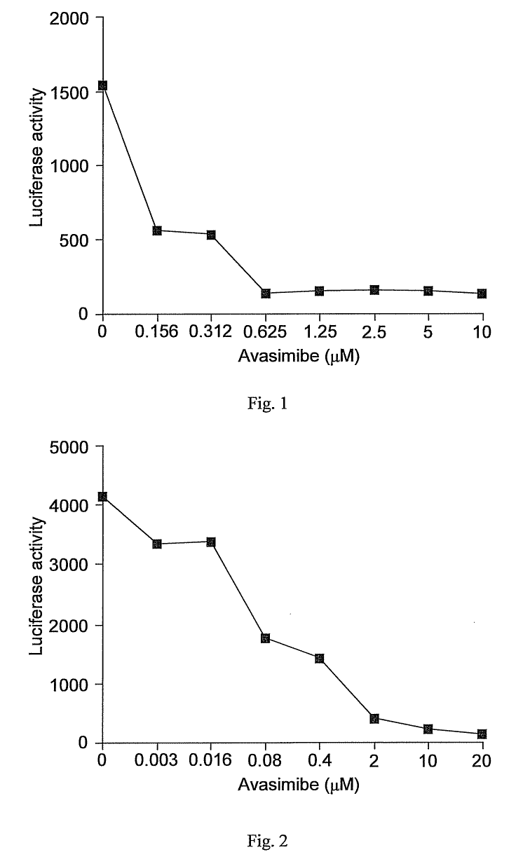

As shown in FIG. 1, avasimibe potently repressed normal hypoxia-induced HRE activation.

The experiment was repeated using conditions as described above with the exception that a broader avasimibe dose response was performed (FIG. 2). This study produced a data set consistent with the original finding and also permitted an estimate of inhibitory potency of avasimibe (IC₅₀ approximately 100 nM) for inhibition of hypoxia-induced HRE activation.

To confirm the effects of avasimibe observed in the above experiments, additional cell types and methods for inducing a hypoxic state were employed. As listed in Table 2, a total of four human cell types and three methods (one environmental and two chemical) for inducing hypoxia were evaluated. In all situations tested, avasimibe exhibited repression of HRE activation.

TABLE 2
Cell types and hypoxia induction methods tested

| Cell type | Hypoxia inducer | HRE activation repression by avasimibe |
|---|---|---|
| A549 | 1% O₂ | yes |
| A549 | CoCl₂ | yes |
| A549 | deferoxamine | yes |
| HeLa | 1% O₂ | yes |
| HeLa | CoCl₂ | yes |
| HeLa | deferoxamine | yes |
| HepG2 | 1% O₂ | yes |
| SH-SY5Y | 1% O₂ | yes |

### Example 2

### Suppression of Hypoxia-Induced BE Activation is a Class Effect of ACAT Inhibitory Compounds

To demonstrate that the effect of avasimibe in suppressing hypoxia-induced HRE activation was not unique to avasimibe and was indicative of a class effect of ACAT inhibitory compounds, a study was conducted using both avasimibe and TMP-153 (N-[4-(2-chlorophenyl)-6,7-dimethyl-3-quinolyl]-N′-(2,4-difluorophenyl)urea, a dual ACAT1 and ACAT2 inhibitor (Sugiyama et al. (1995a) Atherosclerosis 113:71-78; Sugiyama et al. (1995b) Atherosclerosis 118:145-153). For this study, A549 cells were transfected with HRE-luciferase reporter plasmid, and 24 h later cells were seeded in 96-well clear-bottom plates at a density of 1.0×10⁵/ml. Avasimibe or TMP-153 was added at the indicated concentrations and the cells were then exposed for 20 h to a hypoxic biological atmosphere containing 5% CO₂ and 1% O₂ using a Modular Incubator Chamber (Billups-Rothenberg, Inc.). Cells were then lysed and extracts prepared using the Bright Glow Luciferase Assay System (Promega, Inc.) and luciferase activity measurements were obtained in a Veritas luminometer (Promega, Inc.).

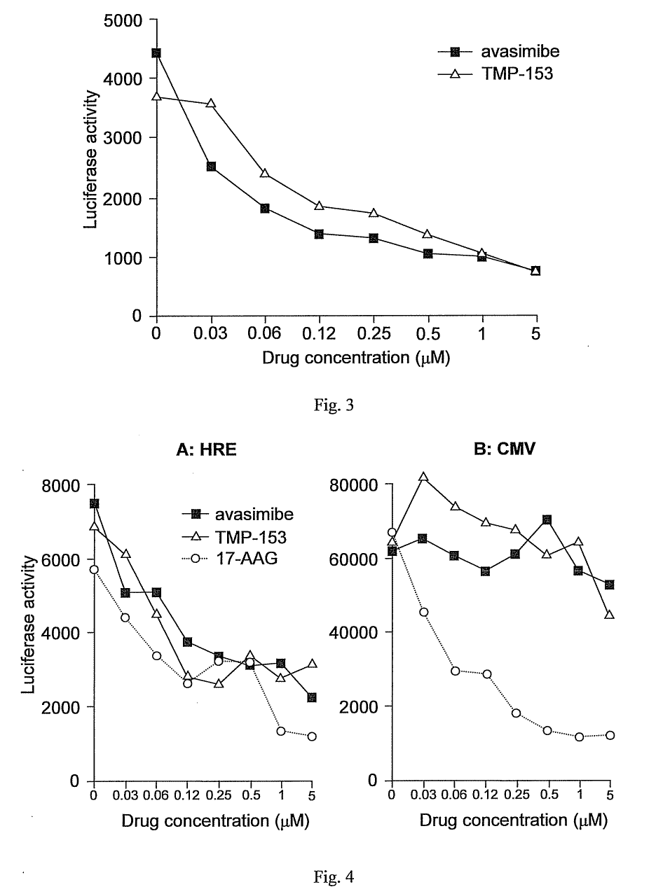

As shown in FIG. 3, the experimental results demonstrate that both avasimibe and TMP-153 can suppress hypoxia-induced HRE activation, a finding supportive of a class effect of ACAT inhibitory compounds, although not necessarily indicative of mechanistic involvement of ACAT, in suppression of the hypoxic response.

### Example 3

### Suppression by ACAT Inhibitory Compounds of Hypoxia-Induced ERE Activation in a Luciferase Reporter Model is not Due to Non-Specific Effects on Luciferase Transcription or Activity

To demonstrate that the suppressive effect of ACAT inhibitory compounds on hypoxia-induced HRE activation in Examples 1 and 2 was not due to an impact on non-HRE mediated luciferase reporter gene transcription or post-transcriptional events, a constitutive cytomegalovirus (CMV) promoter luciferase reporter construct was utilized as a method to detect non-HRE regulated reporter activity. For these experiments, A549 cells were transfected with either the HRE or CMV reporter constructs, and 24 h later the cells were seeded on 96-well clear-bottom plates at a density of 1.0×10⁵/ml. Next day the growth medium was changed and cells were treated with increasing doses of the indicated compounds, which included avasimibe, TMP-153 and 17-(allylamino)-17-demethoxy-geldanomycin (17-AAG), an inhibitor of heat shock protein 90 (HSP-90), a chaperone for the HIF-1 transcription factor (Schulte & Neckers (1998) Cancer Chemother. Pharmacol. 42:273-279). Cells were then exposed for 20 h to a hypoxic biological atmosphere containing 5% CO₂ and 1% O₂ using a Modular Incubator Chamber (Billups-Rothenberg, Inc.). Cells were then lysed and extracts prepared using the Bright Glow Luciferase Assay System (Promega, Inc.) and luciferase activity measurements were obtained in a Veritas luminometer (Promega, Inc.).

As shown in FIG. 4, increasing doses of both avasimibe and TMP-153 strongly suppressed hypoxia-induced HRE activation. However, neither compound exhibited a suppressive effect on the CMV-promoter luciferase reporter activity. In contrast, 17-AAG exhibited a suppressive effect on both the HRE and CMV promoter reporter constructs. These findings show that the ACAT inhibitory compounds avasimibe and TMP-153 do not exhibit indiscriminate effects on either luciferase transcription per se or post-translational luciferase activity, and that their suppressive effect appears to be specific to HRE.

### Example 4

### Effects of Avasimibe on Cell Health and Viability Under Normoxic and Hypoxic Conditions

Additional studies were performed to demonstrate that avasimibe does not induce deleterious effects on cellular health and viability as evaluated by measuring cellular ATP charge. For these experiments A549 cells were plated onto 96-well white-wall clear-bottom plates at a density of 5000 cells/well (panel A) or 2500 cells/well (panel B), and allowed to attach overnight. Cells were then treated with the indicated concentrations of either avasimibe or 17-AAG and then exposed for either 20 h or 65 h to normoxic conditions or a hypoxic atmosphere containing 5% CO₂ and 1% O₂ using a Modular Incubator Chamber (Billups-Rothenberg, Inc.). At the end of drug treatment, cells were subjected to Cell Titer Glo analysis according to manufacturer's instructions (Promega, Inc.) and plates were analyzed using a standard luminometer. Each data point represents the average of triplicate wells.

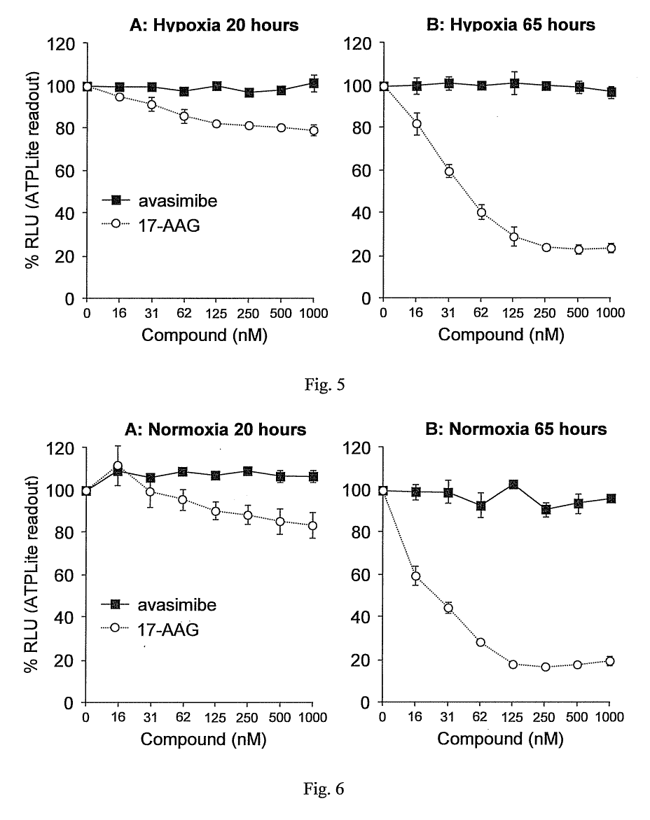

Results demonstrated that avasimibe did not impact cellular ATP charge when measured at either 20 h or 65 h post-dosing under conditions of either hypoxia (FIG. 5) or normoxia (FIG. 6). In contrast, a dose-dependent diminishment of cellular ATP charge was detected at both time points, under both normoxic and hypoxic conditions, in cells treated with 17-AAG. Furthermore, similar results on cell health and viability for both avasimibe and 17-AAG were obtained in additional cell lines tested, including A549, HT1080 and HepG2 cells under conditions of normoxic growth or hypoxic growth induced by 1% O₂ atmosphere or treatment with cobalt chloride (COCl₂).

### Example 5

### Avasimibe can Suppress HRE Activation In Vivo

To demonstrate that avasimibe can suppress or repress a hypoxic response in vivo, a heme oxygenase (HO-1)::luciferase transgenic mouse was used. HO-1 is a key enzyme responsible for metabolism of heme to bilirubin, and increased expression of HO-1 limits tissue damage in response to a wide variety of proinflammatory stimuli associated with oxidative stress including hypoxia. Abnormal HO-1 expression has been associated with a variety of pathological conditions including cancer. The HO-1::luciferase (HO-1::Luc) transgenic line contains 15,000 base pairs of the HO-1 promoter fused to firefly luciferase reporter. This promoter region has several genetic regulatory enhancer sequences including a hypoxia response element (HRE). The HRE response elements of the HO-1 promoter provide sensitivity to changes induced by hypoxia. Cadmium chloride (CdCl₂) is a known inducer of hypoxia in vivo and response to the hypoxic state presumably involves hypoxia-inducible factor 1 (HIF-1) activation of the HRE in the HO-1 promoter. For these studies, whole-body baseline images of HO-1::Luc transgenic mice (n=5 per group) were obtained prior to treatment with avasimibe (0, 3 or 30 mg/kg). Approximately 1 h post-avasimibe treatment, mice were administered CdCl₂ (2 mg/kg, i.v.) and imaged at 4, 6 and 24 h post-CdCl₂ dosing.

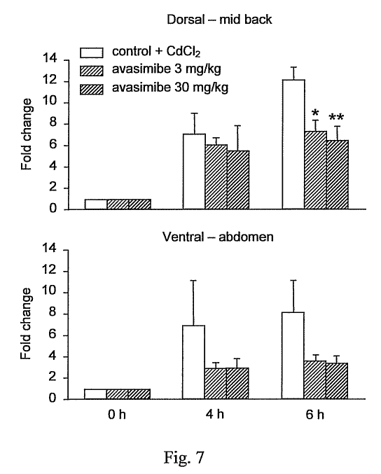

Avasimibe was found to suppress the normal CdCl₂-induced oxidative stress response in the HO-1::Luc transgenic mouse line (FIG. 7). Specifically, avasimibe repressed CdCl₂-induced reporter activation 6 h post-CdCl₂ exposure in the abdominal and mid-back regions of the mice. Additional anatomical regions of the mice also exhibited avasimibe-induced repression of CdCl₂-induced luciferase reporter expression. These findings are consistent with avasimibe suppressing HIF-1 functionality in vivo.

### Example 6

### Avasimibe can Suppress Hypoxia-Induced VEGF Transcript Induction in HeLa Cells

To demonstrate that an ACAT inhibitor could exhibit suppressive effects on the transcriptional regulation of endogenous genes normally induced by hypoxia, effect of avasimibe treatment on hypoxia-induced transcriptional activation of vascular endothelial growth factor (VEGF) was studied. For these studies HeLa cells were plated at a density of 1×10⁶ cells per 10 cm dish, using Dulbecco's modified Eagle's medium (DMEM), and allowed to attach overnight. The medium was aspirated and replaced with fresh DMEM, complete with or without the indicated concentrations of avasimibe, and exposed to hypoxic conditions for 20 h using a Modular Incubator Chamber (atmospheric conditions of 5% CO₂ and 1% O₂ (Billups-Rothenberg, Inc.). Total RNA was then extracted from the pelleted cells using an RNeasy RNA extraction kit (Qiagen) per the manufacturer's protocol. The RNA samples were then analyzed for transcript expression using TaqMan probes from Applied Biosciences, Inc.

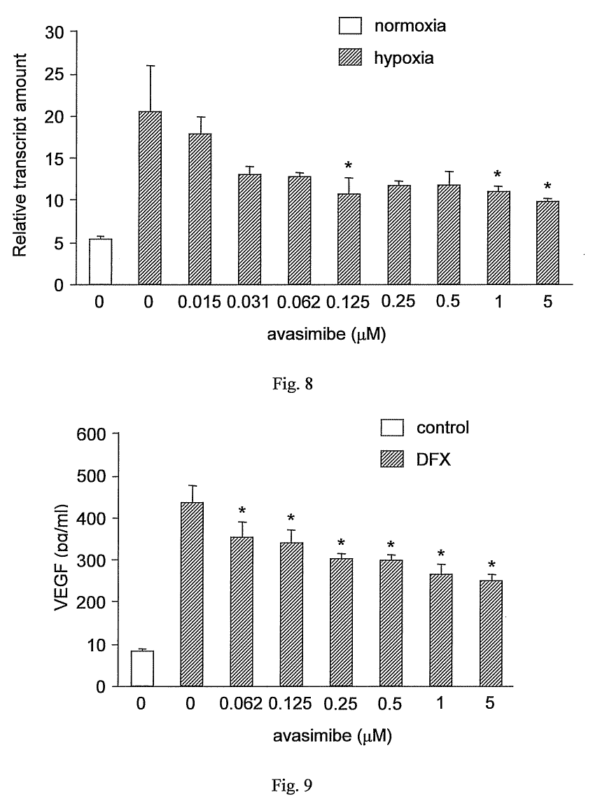

As shown in FIG. 8, HeLa cells exposed to hypoxia in the absence of avasimibe treatment exhibited an approximate 4.5-fold induction of endogenous VEGF transcript as measured by real-time quantitative PCR. Importantly, avasimibe reduced hypoxia-induced VEGF transcript in a concentration-dependent fashion.

Additional studies provided qualitatively comparable results with TMP-153, although this compound did exhibit a marginal but clearly measurable impact on cell health.

### Example 7

### Avasimibe can Reduce the Amount of Detectable VEGF in Conditioned Medium from Hela Cells Grown Under Hypoxic Conditions

Having determined that avasimibe could suppress hypoxia-induced transcriptional activation of VEGF, experiments were next performed to determine whether there was a concomitant effect on production of VEGF protein. For these experiments, HeLa cells were plated in DMEM at 6000 cells/well in a 96-well plate and allowed to recover for approximately 8 h. The medium was then replaced with fresh DMEM containing indicated concentrations of avasimibe with or without 100 μM deferoxamine (Sigma). After 18 h the medium was replaced again with fresh DMEM complete with or without avasimibe and deferoxamine, and 24 h later the supernatant medium was collected and assayed for VEGF using the R&D Systems human VEGF Quantiglo kit, 2nd generation.

As shown in FIG. 9, avasimibe was found to reduce detectable VEGF antigen in the conditioned medium from HeLa cells exposed to hypoxia, in a concentration-dependent fashion.

Additional studies using 1% O₂ atmosphere to induce hypoxia resulted in a quantitatively similar effect on VEGF production.

These studies provide evidence that avasimibe can impact the cellular response to hypoxic stress, specifically production of the pro-angiogenic molecule VEGF. Moreover, TMP-153 produced similar results when the experiments were performed using either deferoxamine or 1% O₂ to induce hypoxia, indicating a class effect of ACAT inhibitory compounds.

### Example 8

### Avasimibe Suppression of VEGF Transcript Induction in HeLa Cells is Restricted to Hypoxic Conditions

As described in Example 7 above, avasimibe can suppress hypoxia-induced transcriptional regulation of endogenous VEGF transcription in HeLa cells. Experiments were therefore performed to address whether avasimibe exhibited similar effects on VEGF transcriptional regulation under normoxic conditions. For these experiments, HeLa cells were grown under either normoxia or hypoxia (atmospheric conditions of 5% CO₂ and 1% O₂) for a total of 20 h in the absence or presence of either avasimibe or echinomycin, an antimicrobial and anti-tumor agent that can inhibit hypoxia-inducible factor 1 (HIF-1) DNA-binding activity (Kong et al. (2005) Cancer Research 65:9047-9055). Total RNA was then extracted from the pelleted cells using an RNeasy RNA extraction kit (Qiagen) per the manufacturer's protocol. The RNA samples were then analyzed for transcript expression using TaqMan probes from Applied Biosciences, Inc.

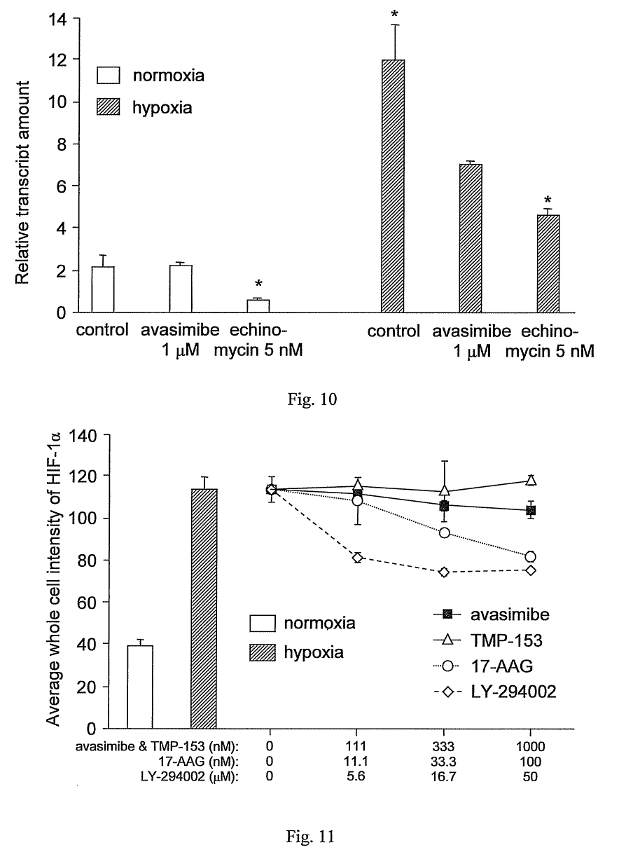

As shown in FIG. 10, avasimibe exhibited minimal to no impact on endogenous VEGF transcript under normoxic conditions, but a clear suppressive effect when cells were grown under hypoxic conditions. In contrast, echinomycin exhibited strong suppressive effects on VEGF transcript levels under conditions of both normoxia and hypoxia. These observations provide further evidence that ACAT inhibitory compounds such as avasimibe can suppress hypoxia-induced VEGF transcript without affecting VEGF levels in normoxic tissues.

### Example 9

### Avasimibe can Suppress Hypoxia-Induced Transcriptional Activation of Genes Important for Broad Cellular Responses to Hypoxia

The above examples demonstrate the suppressive effects of avasimibe on normal hypoxia-induced VEGF transcriptional up-regulation. The cellular transcriptional response requires stabilization and nuclear localization of HIF-1, a heterodimer transcription factor composed of HIF-1α and HIF-1β subunits. Many genes are transcriptionally induced by HIF-1 under hypoxic conditions and promote tumor growth in a variety of ways. These induced genes are involved in glycolysis, angiogenesis, migration, and invasion, all of which are important for tumor progression and metastasis.

Therefore, experiments were performed to determine whether avasimibe could also suppress additional HIF-1 regulated genes normally induced by hypoxia. Candidate genes studied included insulin-like growth factor binding protein 3 (IGF-BP3) and carbonic anhydrase 9 (CA9); the former gene product is important for cell proliferation and survival while the latter is critical for cellular pH regulation. For these studies, HeLa cells were plated in DMEM at a density of 1×10⁶ cells per 10 cm dish and allowed to attach overnight. The medium was aspirated and replaced with fresh DMEM complete with vehicle or 1 μM avasimibe and exposed to hypoxic conditions for 20 h using a Modular Incubator Chamber providing atmospheric conditions of 5% CO₂ and 1% O₂ (Billups-Rothenberg, Inc.). Total RNA was then extracted from the pelleted cells using an RNeasy RNA extraction kit (Qiagen) per the manufacturer's protocol. The RNA samples were then analyzed for transcript expression using primer and probes obtained from Applied Biosciences, Inc.

Results are summarized in Table 3.

TABLE 3
Effect of 1 μM avasimibe on transcriptional
activation of HIF-1 regulated genes

| Transcript | % Suppression |
|---|---|
| VEGF | ~50% |
| IGF-BP3 | ~30% |
| CA9 | ~30% |

### Example 10

### Avasimibe does not Affect Hypoxia-Induced HIF-1α Whole Cell Accumulation or Nuclear Localization

The studies described above characterizing the suppressive effect of avasimibe on hypoxia-induced VEGF transcription indicate that avasimibe is acting to suppress the normal transcriptional response to hypoxia. Mechanistically, the hypoxic response process entails stabilization and nuclear localization of HIF-1, a heterodimer of the HIF-1α and HIF-1β transcription factors (Semenza (2003) Nat. Rev. Cancer 3(10):721-732). In cells growing in a normoxic environment, both HIF-1α and HIF-1β are constitutively expressed; however HIF-1α is subjected to rapid proline hydroxylation and degraded via a proteosome mediated pathway. In contrast, under conditions of hypoxia, the proline hydroxylase activity responsible for tagging HIF-1α is repressed, resulting in stabilization of HF-1α and accumulation of the functional HIF-1 heterodimer.

Therefore, we performed experiments to determine whether avasimibe can suppress either hypoxia-induced HIF-1α stabilization or subsequent nuclear translocation. For the former study, A549 cells were plated onto 96-well BD poly-(D-lysine) coated, black-wall clear-bottom plates at a density of 5000 cells/well, and allowed to attach for 6 h. The indicated drugs were added to achieve the final concentrations, and cells were grown in the presence or absence of 250 μM deferoxamine in growth medium. After 20 h, the cells were fixed with pre-warmed 3.7% paraformaldehyde for 10 min at 37° C., washed with PBS, and stained with BD anti-HIF-1α monoclonal antibody in 1% donkey serum PBS for 4 h, washed 6 times with PBS, incubated with anti-mouse Alexa Fluor 488 conjugated secondary antibody and Hoechst nuclear dye, washed 6 times with PBS, and 200 μl PBS was kept in each well in the end. Cell images were acquired with Cellomics ArrayScan 4.5 and quantified with Compartmental Analysis Bio-application software. The total cellular intensity (combined cytoplasmic and nuclear intensity) of HIF-1α was used as a parameter for the total amount of HIF-1α in the cell.

Results are presented in FIG. 11. Each data point represents the average of triplicate wells.

Similar results were obtained with the ACAT inhibitory compound TMP-153. In contrast, LY-294002, a phosphoinositide 3 (PI3) kinase inhibitor, and 17-AAG, an HSP-90 inhibitor, diminished the whole cell accumulation of HIF-1α, consistent with previous reports describing effects of these compounds on HIF-1α stability under hypoxic conditions (as reviewed by Semenza (2003), supra).

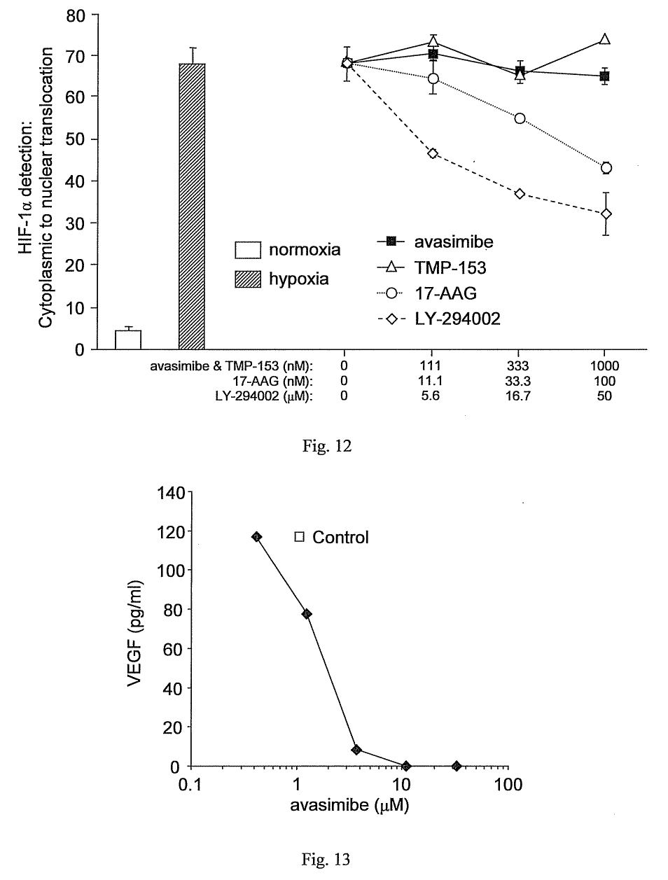

Re-examination of the data set from these studies using a different set of scoring parameters allowed a determination as to whether avasimibe could suppress nuclear accumulation of HIF-1α. As shown in FIG. 12, no evidence was found for diminished HIF-1α antigen in the nuclear compartment of cells when exposed to avasimibe.

Similar results were obtained with TMP-153. In contrast, LY-294002 and 17-AAG diminished nuclear accumulation of HIF-1α, consistent with previous reports describing effects of these compounds on HIF-1α stability under hypoxic conditions (as reviewed by Semenza (2003), supra). Additional experiments were also performed in several other cell lines, specifically HeLa and HT-1080. These studies confirmed observations in A549 cells that avasimibe does not suppress either whole cell accumulation or nuclear translocation of HIF-1α.

### Example 11

### Avasimibe can Suppress the Amount of Detectable VEGF in Conditioned Medium from Colon 26 Cells Grown Under Hypoxic Conditions

It having been demonstrated that avasimibe can suppress hypoxia-induced production of VEGF protein in HeLa cells, a study was conducted to determine whether avasimibe could have effects on hypoxia-induced VEGF secretion from a classic tumor-inducing cell line such as colon 26, a well-characterized mouse cell line derived from an undifferentiated adenocarcinoma. Colon 26 tumor cells were obtained from National Cancer Institute, Frederick, Md. For these experiments, the cells were plated in 96-well plates at a density of 15,000 cells per well and allowed to attach to the plate by incubating for 3 h at 37° C. under normoxic conditions. Cells were then pre-incubated in the presence of avasimibe for 1 h. Hypoxia was simulated by addition of 75 μM CoCl₂ and the cells were incubated for 24 h at 37° C., 5% CO₂ (atmospheric O₂ levels). VEGF levels secreted into the medium were measured in duplicate samples by ELISA using R&D Systems human VEGF Quantiglo kit, 2nd generation.

As shown in FIG. 13, avasimibe suppressed detectable VEGF antigen in the conditioned medium from colon 26 cells exposed to hypoxia, in a concentration-dependent fashion. For comparative purposes, VEGF levels measured in the absence of any test compound treatment are indicated by the control.

### Example 12

### Avasimibe can Slow Tumor Growth in Mice

As shown in Example 11 above, avasimibe exhibits a concentration-dependent effect on hypoxia-induced VEGF secretion from colon 26 cells. Importantly, colon 26 cells injected subcutaneously in the mouse form a rapidly growing vascularized tumor mass that induces cachexia. Experiments were therefore performed to evaluate whether avasimibe could suppress tumor growth and tumor and volume in vivo.

For these studies, female Balb/c mice were obtained at 6-8 weeks of age from Charles River Laboratories (Wilmington, Mass.). The test compound avasimibe was added at the appropriate concentration to 0.5% methylcellulose, 0.025% polysorbate 80 in deionized water to form a suspension, and mice were dosed orally (p.o.) twice daily (b.i.d.) with 100 μl of suspension per mouse per dose. Colon 26 tumor cells were obtained from National Cancer Institute, Frederick, Md. Cells were grown in RPMI with 10% FCS and 2 mM L-glutamine. Tumor cells were harvested from sub-confluent cultures, washed in RPMI (no phenol red), and resuspended in RPMI (no phenol red) at 5×10⁷ cells/ml. Tumor cells were kept in culture for 4 weeks or less prior to usage. The mice were acclimated in a barrier care facility caged in groups of five. Mice were maintained on standard Purina mouse chow and had free access to water. Mice were injected subcutaneously in the flank with 5×10⁶ tumor cells per mouse. Tumor volumes were measured at intervals over the course of the experiment. When tumors were of a size consistent with a weight of 50-100 mg, 5-7 days post-implantation, animals were sorted into groups consisting of 10 mice per group and dosing was initiated. Dosing was by oral gavage and included vehicle-treated or avasimibe-treated at either 10 mg/kg b.i.d. or 100 mg/kg b.i.d. Tumors were measured with a caliper, and volumes were determined using the formula: tumor volume=width²×length×0.52. On day 21 of the study, the mice were sacrificed by CO₂ euthanasia, and the tumors were excised and weighed.

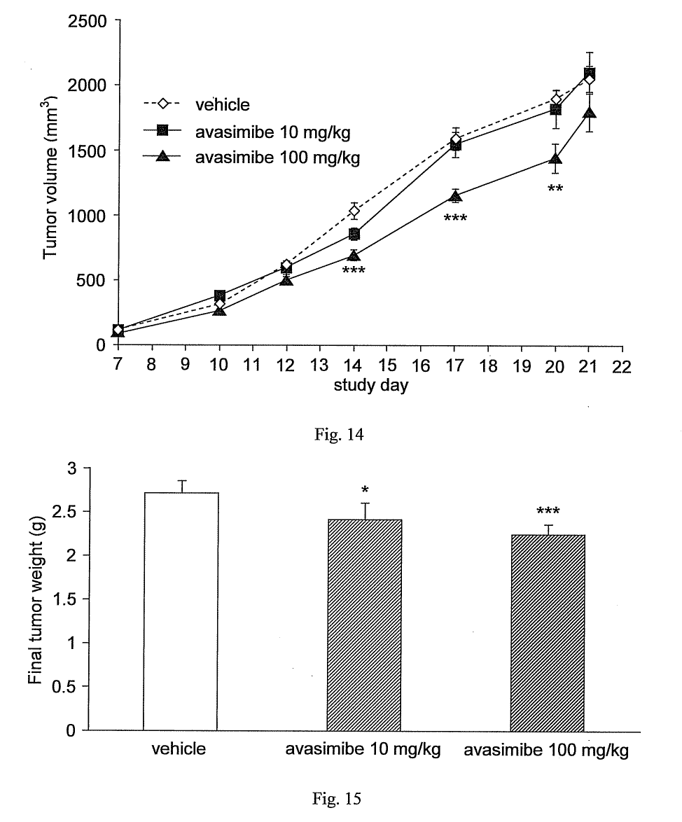

As shown in FIG. 14, avasimibe dose-dependently slowed progression of tumor growth during the course of the study, with a significant effect observed at the higher dose of avasimibe. For the tumor volume measurements repeatedly taken over the duration of the study, a repeated measurement analysis of variances (ANOVA) model, specifically analysis of response profile model, was used for assessing the change and comparing the mean profiles of the treatment groups in tumor volume over the study course. The repeated ANOVA model was conducted using PROC MIXED procedure in SAS (SAS Inc. Cary, N.C.).

In addition to the measured effects of avasimibe on tumor growth, avasimibe also exhibited a dose-dependent significant effect on final tumor weight (FIG. 15). For these studies, a one-way or one-factor analysis of variances (ANOVA) model was applied on final tumor weights of the mice from the four treatment groups. Following the overall significant F-test, differences among the treatment groups were assessed by pairwise comparisons.

These studies demonstrate that avasimibe can inhibit tumor growth in vivo and are consistent with avasimibe altering the necessary vascularization requirement of a solid tumor.

All patents and publications cited herein are incorporated by reference into this application in their entirety.

The words “comprise”, “comprises”, and “comprising” are to be interpreted inclusively rather than exclusively.

What is claimed is:

1. A method for treating a solid tumor in a subject, comprising administering to the subject an ACAT inhibitory compound or a prodrug thereof, wherein (a) the solid tumor is at least about 2 mm in diameter and (b) the compound or prodrug thereof is administered in an amount that is therapeutically effective, but ineffective to cause unacceptable toxicity to normoxic tissues.

2. The method of claim 1, wherein the subject has a cancerous or precancerous condition.

3. The method of claim 2, wherein the condition is selected from the group consisting of
acinar adenocarcinoma,
acinar carcinoma,
acral-lentiginous melanoma,
actinic keratosis,
adenocarcinoma,
adenocystic carcinoma,
adenosquamous carcinoma,
adnexal carcinoma,
adrenal rest tumor,
adrenocortical carcinoma,
aldosterone-secreting carcinoma,
alveolar soft part sarcoma,
amelanotic melanoma,
ameloblastic carcinoma,
ampullary carcinoma,
anal canal cancer,
anaplastic thyroid carcinoma,
angiosarcoma, apocrine carcinoma,
Askin's tumor,
astrocytoma,
basal cell carcinoma,
basaloid carcinoma,
basosquamous cell carcinoma,
biliary cancer,
bone cancer,
bone marrow cancer,
botryoid sarcoma,
brain cancer,
breast cancer,
bronchioalveolar carcinoma,
bronchogenic adenocarcinoma,
bronchogenic carcinoma,
carcinoid,
carcinoma en cuirasse,
carcinoma ex pleomorphic adenoma,
cervical cancer,
chloroma,
cholangiocellular carcinoma,
chondrosarcoma,
choriocarcinoma,
choroid plexus carcinoma,
clear cell adenocarcinoma,
colon cancer,
colorectal cancer,
comedocarcinoma,
cortisol-producing carcinoma,
cylindrical cell carcinoma,
dedifferentiated liposarcoma,
ductal adenocarcinoma of the prostate,
ductal carcinoma,
ductal carcinoma in situ,
duodenal cancer,
eccrine carcinoma,
embryonal carcinoma,
endometrial carcinoma,
endometrial stromal sarcoma,
endometrioid adenocarcinoma,
endometrioid carcinoma,
epithelioid sarcoma,
esophageal cancer,
Ewing's sarcoma,
exophytic carcinoma,
fibroblastic sarcoma,
fibrocarcinoma,
fibrolamellar carcinoma,
fibrosarcoma,
follicular thyroid carcinoma,
gallbladder cancer,
gastric adenocarcinoma,
giant cell carcinoma,
giant cell sarcoma,
giant cell tumor of bone,
granulosa cell carcinoma,
head and neck cancer,
hemangioma,
hemangiosarcoma,
hepatocellular carcinoma,
Hürthle cell carcinoma,
ileal cancer,
infiltrating lobular carcinoma,
inflammatory carcinoma of the breast,
intraductal carcinoma,
intraepidermal carcinoma,
jejunal cancer,
Kaposi's sarcoma,
Krukenberg's tumor,
Kulchitsky cell carcinoma,
Kupffer cell sarcoma,
large cell carcinoma,
larynx cancer,
lentigo maligna melanoma,
liposarcoma,
liver cancer,
lobular carcinoma,
lobular carcinoma in situ,
lung cancer,
lymphoepithelioma,
lymphosarcoma,
malignant melanoma,
medullary carcinoma,
medullary thyroid carcinoma,
meningeal carcinoma,
Merkel cell carcinoma,
micropapillary carcinoma,
mixed cell sarcoma,
mucinous carcinoma,
mucoepidermoid carcinoma,
mucosal melanoma,
myxoid liposarcoma,
myxosarcoma,
nasopharyngeal carcinoma,
nodular melanoma,
non-clear cell renal cancer,
non-small cell lung cancer,
oat cell carcinoma,
ocular melanoma,
oral cancer,
osteoid carcinoma,
osteosarcoma,
ovarian cancer,
Paget's carcinoma,
pancreatic cancer,
papillary adenocarcinoma,
papillary carcinoma,
papillary thyroid carcinoma,
pelvic cancer,
periampullary carcinoma,
phyllodes tumor,
pituitary cancer,
pleomorphic liposarcoma,
preinvasive carcinoma,
primary intraosseous carcinoma,
prostate cancer,
rectal cancer,
renal cell carcinoma,
rhabdomyosarcoma,
round cell liposarcoma,
scar cancer,
schistosomal bladder cancer,
schneiderian carcinoma,
sebaceous carcinoma,
signet-ring cell carcinoma,
skin cancer,
small cell lung cancer,
small cell osteosarcoma,
soft tissue sarcoma,
spindle cell carcinoma,
spindle cell sarcoma,
squamous cell carcinoma,
stomach cancer,
superficial spreading melanoma,
synovial sarcoma,
telangiectatic sarcoma,
terminal duct carcinoma,
testicular cancer,
thyroid cancer,
transitional cell carcinoma,
tubular carcinoma,
tumorigenic melanoma,
undifferentiated carcinoma,
urachal adenocarcinoma,
urinary bladder cancer,
uterine cancer,
uterine corpus carcinoma,
uveal melanoma,
vaginal cancer,
verrucous carcinoma,
villous carcinoma,
well-differentiated liposarcoma,
Wilm's tumor,
yolk sac tumor,
and combinations thereof.

4. The method of claim 1, wherein the solid tumor is malignant.

5. The method of claim 1, wherein the ACAT inhibitory compound or prodrug thereof is administered in an amount effective to slow, retard, arrest or reverse growth of the tumor.

6. The method of claim 5, wherein the effect on tumor growth is associated with inhibition of angiogenesis.

7. The method of claim 6, wherein the inhibition of angiogenesis is associated with suppression of hypoxia-induced activation of at least one proangiogenic signaling factor.

8. The method of claim 7, wherein the at least one signaling factor comprises VEGF.

9. The method of claim 5, wherein the effect on tumor growth is associated with inhibition of tumor cell proliferation.

10. The method of claim 9, wherein the inhibition of angiogenesis is associated with suppression of hypoxia-induced activation of at least one signaling factor that promotes tumor cell proliferation.

11. The method of claim 10, wherein the at least one signaling factor comprises IGF-BP3.

12. The method of claim 1, wherein the compound is selected from the group consisting of
acaterin,
AD-6591,
avasimibe,
bezafibrate,
AS-183,
AS-186,
BW-447A,
CI-976
CI-999,
CL-277082,
CL-283546,
CL-283796,
colestyramine,
CP-105191,
CP-113818,
crepiside I,
crilvastatin,
cyclandelate,
E-5324,
EAB-309,
eflucimibe,
eldacimibe,
epicochlioquinone A,
F-1394,
F-12511,
FCE-25390,
FCE-27677,
FCE-28645A,
FR-129169,
FR-145237,
FR-186054,
FY-087,
GERI-BP-001M,
GERI-BP-002A,
glibenclamide,
glisoprenins,
GW-447C88,
gypsetin,
ixerin M,
K-604,
K-9406,
K-10085,
KW-3033,
KY-331,
KY-455,
lateritin,
lecimibide,
LS-3115,
melinamide,
naringenin,
NTE-122,
P-06139,
pactimibe,
PD-132301-2,
PD-138142-15,
purpactins,
pyripyropenes,
R-106578,
RP-64477,
RP-60676,
RP-73163,
Sandoz 57-118,
Sandoz 58-035,
SC-435,
SCH-48461,
SKF-98016,
SKF-99085,
SMP-500,
SMP-797,
SR-9223i,
T-2591,
tamoxifen,
TEI-6522,
TEI-6620,
TMP-153,
TS-962,
U-73482,
U-76807,
ulmoidol,
VULM-1457,
YM-17E,
YM-750,
acyclic (diphenylethyl)diphenylacetamides,
N-alkyl-N-biphenylylmethyl-N′-arylureas and derivatives thereof,
N-alkyl-N-[(fluorophenoxy)benzyl]-N′-arylureas and derivatives thereof,
N-alkyl-N-(heteroaryl-substituted benzyl)-N′-arylureas and derivatives thereof,
2-(alkylthio)-4,5-diphenyl-1H-imidazole derivatives,
amides of 1,2-diarylethylamines and derivatives thereof,
N-[1-butyl-4-[3-[3-(hydroxy)propoxy]phenyl]-1,2-dihydro-2-oxo-1,8-naphthyridin-3-yl]-N′-(2,6-diisopropyl-4-aminophenyl)urea,
N-chlorosulfonyl isocyanate and derivatives thereof,
27-cis-p-coumaroyloxyursolic acid,
27-trans-p-coumaroyloxyursolic acid,
cyclic sulfides derived from hetero-Diels-Alder reaction of thioaldehydes with 1,3-dienes,
diaryl-substituted heterocyclic ureas and derivatives thereof,
N-(2,2-dimethyl-2,3-dihydrobenzofuran-7-yl)amide derivatives,
2-(1,3-dioxan-2-yl)-4,5-diphenyl-1H-imidazoles and derivatives thereof,
N-(4,5-diphenylthiazol-2-yl)alkanamides and derivatives thereof,
N-(4,5-diphenylthiazol-2-yl)-N′-aryl(thio)ureas and -alkyl(thio)ureas and derivatives thereof,
2,6-disubstituted-3-imidazolylbenzopyrane derivatives,
fatty acid anilides,
heterocyclic amides and derivatives thereof,
hydroxyphenylurea derivatives,
23-hydroxyursolic acid,
indoline derivatives with an amide or urea moiety,
N-(4-oxochroman-8-yl)amide derivatives,
N-phenyl-6,11-dihydrodibenz[b,e]oxepin-11-carboxamides and derivatives thereof,
polyacetylene analogs,
3-quinolylurea derivatives,
short-chain ceramide and dihydroceramide,
terpendoles,
tetrazole amide derivatives of (+/−)-2-dodecyl-alpha-p-phenyl-N-(2,4,6-trimethoxy-phenyl)-2H-tetrazole-5-acetamide,
4,4-bis(trifluoromethyl)imidazolines and derivatives thereof,
triterpenes and derivatives thereof,
prodrugs thereof, and combinations thereof.

13. The method of claim 1, wherein the compound is avasimibe.

14. The method of claim 1, wherein the subject is an adult human and the compound or prodrug thereof is administered in a dosage amount of about 1 to about 5000 mg/day.

15. The method of claim 1, wherein the compound or prodrug thereof is administered systemically.

16. The method of claim 15, wherein administration is via an oral, rectal, nasal, transmucosal, intrapulmonary, intravenous, intraperitoneal, intramuscular, subcutaneous, intradermal or transdermal route.

17. The method of claim 1, wherein the compound or prodrug thereof is administered to the locus of the tumor by topical application, local injection or surgical implantation.

18. The method of claim 1, wherein the administration of the compound or prodrug thereof is a component of an anti-cancer regimen further comprising one or more of surgery, radiation therapy or administration of one or more drugs other than an ACAT inhibitory compound or prodrug thereof.

19. A method for reducing tumor growth and/or metastasis in a subject having a cancerous or precancerous condition, comprising (a) determining presence of one or more solid tumors having a diameter of at least about 2 mm in tissue of the subject, and (b) if one or more solid tumors having a diameter of at least about 2 mm are determined to be present, administering to the subject an ACAT inhibitory compound or a prodrug thereof in an amount that is therapeutically effective, but ineffective to cause unacceptable toxicity to normoxic tissues.

20. The method of claim 19, wherein said determining comprises examining a tissue sample from the subject obtained by excision or biopsy, or an image of tissue obtained by endoscopy, X-ray, CT or MRI.

21. A method for treating a hypoxia-induced condition in a subject, comprising administering to the subject an ACAT inhibitory compound or a prodrug thereof, wherein the compound or prodrug thereof is administered in an amount that is therapeutically effective, but ineffective to cause unacceptable toxicity to normoxic tissues.
